(19) United States
(12) Patent Application Publication
(10) Pub. No.: US 2017/0150904 A1
(43) Pub. Date: Jun. 1, 2017

(54) METHOD FOR MEASURING SIZE OF LESION WHICH IS SHOWN BY ENDOSCOPE, AND COMPUTER READABLE RECORDING MEDIUM

(71) Applicants: Hyun Jun PARK, Gyeonggi-do (KR); Ji Yong AHN, Seoul (KR); Ji Suk LEE, Gyeonggi-do (KR)

(72) Inventors: Hyun Jun PARK, Gyeonggi-do (KR); Ji Yong AHN, Seoul (KR); Ji Suk LEE, Gyeonggi-do (KR)

(21) Appl. No.: 15/310,385

(22) PCT Filed: May 18, 2015
(86) PCT No.: PCT/KR2015/004945

§ 371 Date: Nov. 10, 2016

(30) Foreign Application Priority Data
May 20, 2014 (KR) 10-2014-0060343

(51) Int. Cl. A61B 5/107; A61B 1/04; G06T 7/60; A61B 5/00; A61B 10/04; G06T 7/00; A61B 1/00; A61B 5/103

(52) U.S. Cl. CPC A61B 5/1079; A61B 1/00009; A61B 1/0005; A61B 1/04; A61B 5/1032; A61B 5/1076; A61B 5/743; A61B 10/04; G06T 7/0012; G06T 7/60; G06T 2207/10068; G06T 2207/30096

(57) ABSTRACT

A method is disclosed for measuring size of lesion from an image photographed by using a photographing device such as an endoscope. An image shown with lesion is shown on a screen, a lesion area is confirmed from the image shown on the screen, and the size of lesion area is calculated and displayed based on designated scale, whereby the size of lesion can be accurately provided, which is conducive to medical doctors in diagnosis and treatment.

### TECHNICAL FIELD

The teachings in accordance with exemplary and non-limiting embodiments of this invention relate generally to a method for measuring size of lesion which is shown by endoscope, and more particularly, to a method for measuring size of lesion which is shown by endoscope, whereby the size of lesion can be accurately calculated and displayed from an image photographed by using a photographing device including an endoscope.

### BACKGROUND ART

Absence and/or presence of diseases, and states of the diseases are ascertained using photographing devices including endoscopes in various medical fields. In general, information on size of lesion on an endoscope acts as an important factor in determining diagnosis and treatment direction of diseases. For example, cancer or adenoma found by an endoscope can be determined in treatment and method using the endoscope depending on sizes of the cancer and adenoma.

When the exact sizes of various lesions after operations are known, treatment methods such as endoscopic treatment, medicine treatment or passage observation can be determined, or the exact sizes of various lesions can be conducive to determination of treatment effect including grasp of changes in lesions.

Furthermore, treatments including operations may be required in response to changes in sizes of submucosal tumor, such that it is important to measure the exact size of tumor. In addition, the degree of treatment effect of ulcer can be learned in response to size of ulcer and changes in size of the ulcer after treatment, whereby necessity of additional administration of medicine can be evaluated.

However, there are currently few methods for measuring an actual size of an image observed on an endoscope used in the clinic, and therefore, the current situation is that it is not easy to grasp the exact size of lesion and exact opinion.

### DISCLOSURE OF INVENTION

### Technical Problem

Accordingly, the present invention is disclosed in consideration of the above-mentioned problems and it is an object of the present invention to provide a method for measuring size of lesion which is shown by endoscope, whereby the size of lesion can be accurately calculated and displayed from an image photographed by using a photographing device including an endoscope.

### Solution to Problem

In one general aspect of the present invention, there is provided a method for measuring size of lesion which is shown by endoscope, the method comprising:

displaying, on a screen, an image shown with a lesion which is shown by an endoscope while being performed by a computer program executed by a computer device; determining a lesion area of the image displayed on the screen, and a scale (a unit length on the image corresponding to an actual unit length); and calculating a size of the determined lesion area using the scale.

Preferably, but not necessarily, the method may further comprise displaying the calculated size of the lesion area on the screen.

Preferably, but not necessarily, the determination of the lesion area may be realized by color comparison.

Preferably, but not necessarily, the color comparison may be performed only within an analysis object area inputted by a user.

Preferably, but not necessarily, the determination of the lesion area may be performed based on an area inputted by a user.

Preferably, but not necessarily, the method may further comprise receiving from a user information for calculating a unit length on the image.

Preferably, but not necessarily, the image displayed on the screen may be an image photographed along with a wire connected to a biopsy tool, and the computer program may provide a function of allowing a user to draw an outline to the lesion area along a lengthwise direction of the wire, and a function of allowing the outline to designate two points touched by the lesion area, and may recognize a length between the designated two points as a unit length of the image.

Preferably, but not necessarily, the size of the lesion area may be calculated by using the number of square lattices entered into the lesion area, where length of each side is a unit length of the image.

Preferably, but not necessarily, the method may further comprise calculating a major axis length and a minor axis length of the determined lesion area, or one of the major axis length and the minor axis length of the determined lesion area, based on the scale.

Preferably, but not necessarily, major axis and minor axis may be recognized by receiving information that designates a relevant axis.

In another general aspect of the present invention, there is provided a computer readable recording medium, wherein the computer readable recording medium is recorded with a program capable of executing the method for measuring size of lesion, which is shown by endoscope, and a computer device can perform the method for measuring size of lesion which is shown by endoscope by executing the program recorded on the record medium.

### Advantageous Effects of Invention

The present invention has an advantageous effect in that size of the lesion can be accurately and quickly calculated from an image photographed by using a photographing device such as an endoscope, which is conducive to medical doctors in diagnosis and treatment.

### BRIEF DESCRIPTION OF DRAWINGS

FIG. 1 is a schematic block diagram illustrating an example of a diagnosis device using an endoscope.

FIG. 2 is a schematic example of a user interface screen provided by an image processing program.

FIG. 3 is a method for measuring size of lesion which is shown by endoscope according to an exemplary embodiment of the present invention.

FIG. 4 is a schematic view illustrating an analysis object area according to an exemplary embodiment of the present invention.

FIG. 5 is a schematic view illustrating direct designation of a lesion area by a user according to an exemplary embodiment of the present invention.

FIGS. 6 and 7 are schematic views illustrating designation of a unit length using an image of a wire connected to a biopsy tool according to an exemplary embodiment of the present invention.

FIG. 8 is a schematic view illustrating calculation of size of lesion using a square lattice according to an exemplary embodiment of the present invention.

FIG. 9 is a schematic view illustrating a user interface screen displayed with information on calculated size of legion area, major axis length and minor axis length according to an exemplary embodiment of the present invention.

### BEST MODE FOR CARRYING OUT THE INVENTION

Hereinafter, example embodiments of the present invention will be described in more detail with reference to the accompanying drawings.

The method for measuring size of lesion which is shown by endoscope according to the present invention is performed by an image processing program performed by a computer system (device). At this time, the computer system may be any types of computers including personal computers, mobile computers, tablet computers, and diagnosis-dedicated terminals. Furthermore, the image processing program is defined by a computer program configured to execute the method for measuring size of lesion which is shown by endoscope according to the present invention regardless of its name. The image processing program may be variably embodied by being configured to be independently executed, by being configured as a module of another program, or being configured to be called by another program in a distributed format.

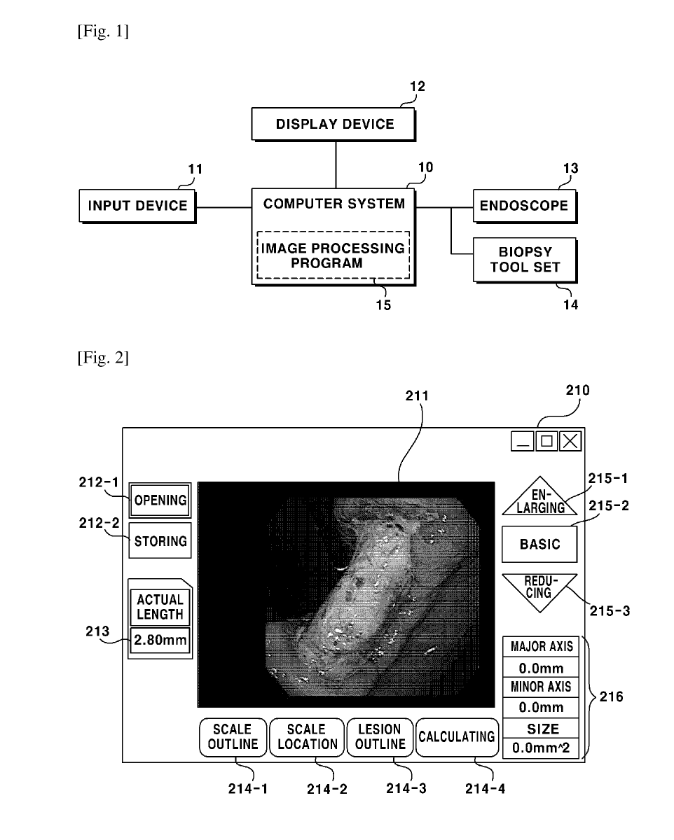

FIG. 1 is a schematic block diagram illustrating an example of a diagnosis device using an endoscope, where a computer system (10) may be connected by various peripheral devices including a display device (12), an input device (11), an endoscope (13), and a biopsy tool set (14), and an image processing program (15) may be installed for processing an image photographed by the endoscope (13).

A user (medical doctor) may perform a diagnosis using the endoscope (13) and the biopsy tool set (14), and may observe or store the image photographed by the endoscope (13). In general, the biopsy tool set (14) is configured to be connected to a wire.

The image processing program (15) is so configured as to allow a user to manage a user-photographed image through the endoscope (13). The image processing program (15) may be operated by a command inputted from various input devices (11) including a mouse, a keyboard, and a touch panel, and may display or store the image photographed through the endoscope (13) through the display device (12). The image processing program (15) may be variably configured as occasion demands, and particularly, may measure size of lesion according to the present invention.

FIG. 2 is a schematic example of a user interface screen (210) provided by an image processing program (15), where an image photographed by the endoscope (13) is displayed on an image display window (211), and a user can call an image stored in the computer system (10) by clicking an open button (212-1), and may store a current screen by clicking a storage button (212-2).

Furthermore, the image of the image display window (211) may be enlarged or reduced by clicking an enlargement button (215-1) or a reduction button (215-3), and may be displayed in an original size by clicking a basic button (215-2). It should be apparent that FIG.2 is an example for explanation, and the user interface screen can be variably configured as many as desired, as occasion demands.

FIG. 3 is a method for measuring size of lesion which is shown by endoscope according to an exemplary embodiment of the present invention.

First, the image processing program (15) may display a lesion-showing image on the image display window (211) by loading the lesion-showing image in response to a user command (S111). The image displayed on the image display window (211) at step S111 may be an image currently photographed by the endoscope (13), or may be one of images selectively called that are stored by a user in the computer system (10).

Furthermore, the image processing program (15) may determine a lesion area and scale of the image displayed on the screen (S112). The determination of the lesion area on the image displayed on the screen may be realized by clicking a lesion outline button (214-3) and may determine the lesion area on the image displayed on the image display window (211). The determination of lesion area at step S112 may be realized by variable methods.

For example, in view of the fact that a color of lesion area is different from a color of a normal area, an out-boundary of the lesion area may be determined by color comparison of images shown on a screen by the image processing program. At this time, color information determined as lesion area may be set up by various methods, such as prior storage of the color information, input of a color scope by a user, or designation by click of, through a mouse, several positions of areas determined as lesion areas.

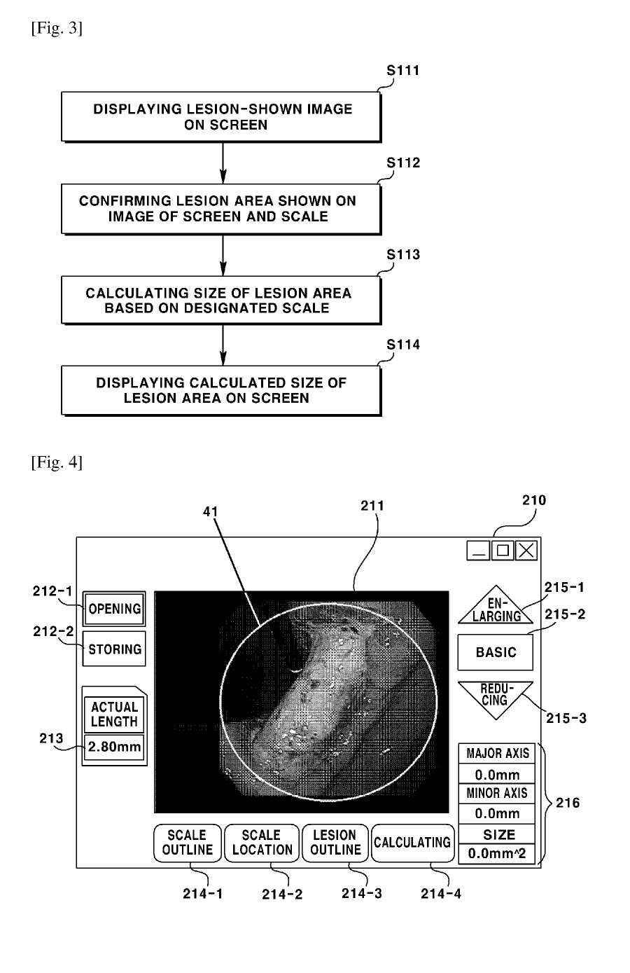

Then, the image processing program (15) may determine an out-boundary of the lesion area by investigation of a scope having a color within a predetermined scope similar to a designated color. With this exemplary embodiment, as one of faster methods for execution, configuration may be made in such a manner that the image processing program (15) receives an analysis object area from a user, and color comparison is performed only within the inputted analysis object area. FIG.4 is a schematic view illustrating an analysis object area in a round shape (41) according to an exemplary embodiment of the present invention.

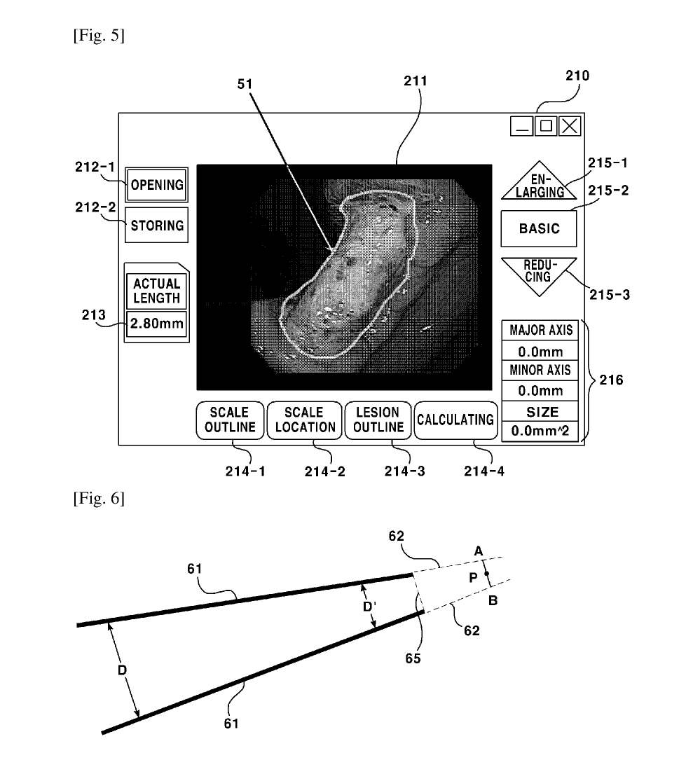

As a still another method for determining a lesion area at step S112, the image processing program (15) may determine a lesion area by receiving, from a user, information directly designating the lesion area. That is, the user can directly designate an out-boundary of an area determined as a lesion using the input device (11) such as a mouse. FIG. 5 is a schematic view illustrating a direct designation by a user of an outboundary (51) of a lesion area according to an exemplary embodiment of the present invention.

The term of “scale” in S112 defines a unit length on an image corresponding to an actual unit length. That is, size of an image displayed on a screen of the display device (12) is different from the actual size, such that it is necessary to match the length on the image to an actual unit length corresponding to the length of the image to a certain degree.

The scale may be automatically designated according to photographed state of image, and may be configured by receiving information from a user for calculating an actual unit length or a unit length on the image corresponding to the actual unit length.

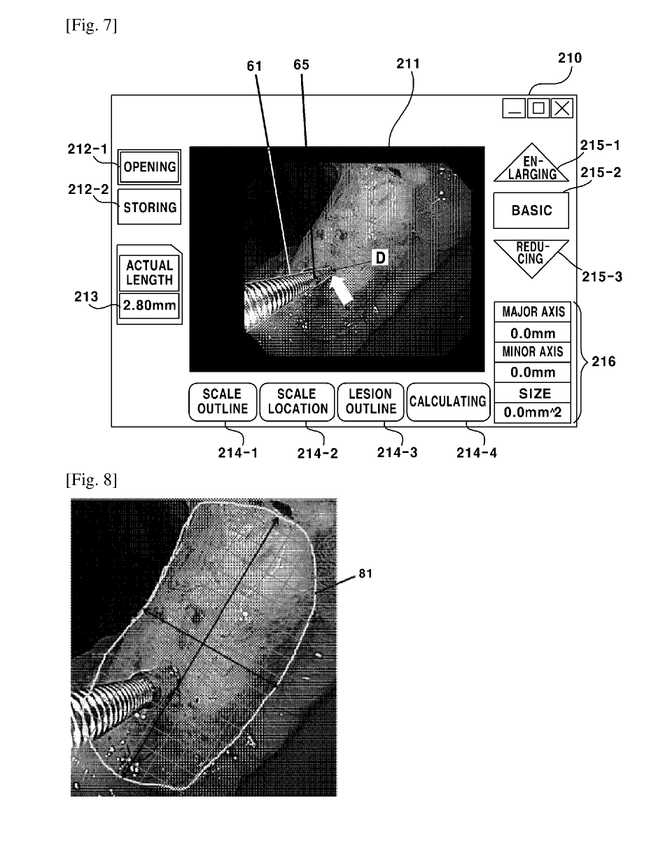

FIGS. 6 and 7 are schematic views illustrating designation of a unit length using an image of a wire connected to a biopsy tool according to an exemplary embodiment of the present invention.

Referring to FIGS. 6 and 7, a detailed exemplary embodiment receiving, from a user, information for calculating a unit length on an image will be explained.

At this time, an image shown with a lesion is an image photographed along with a wire connected to a biopsy tool. Although the biopsy tool formed at a distal end of the wire may vary in terms of types, sizes and shapes, it is preferable to use a wire body diameter based on scale standard because most of the wires are constant in body diameter.

However, even if an actual wire body diameter is constant (D=D′), wire images photographed by an endoscope do not show the same length depending on distance as illustrated in FIG. 6.

Because of this inconsistency, in order to apply an accurate scale to a lesion area, it is important to grasp a wire body diameter at points (A, B) where a wire of lengthwise outline (61) passes a distal end (65) of the wire, is extended (62) and touches the lesion area. To this end, a user clicks a scale outline button (214-1), and draws a wire lengthwise outline (61) using a mouse up to A and B points.

Furthermore, the user designates a wire body diameter at the lesion area by clicking a scale position button (214-2). The wire body diameter may be designated by various methods including by clicking A and B points in FIG. 6, or drawing a line connecting A and B points. At this time, an image may be enlarged in order to accurately and conveniently designate the wire body diameter, whereby the image processing program (15) may recognize the wire body diameter designated by the user as a unit length on the image.

Meantime, the image processing program (15) may be so configured as to automatically calculate the wire body diameter at the measurement points as the user designates the wire lengthwise outline (61) and the measurement point (P), where the measurement point (P) is an arbitrary point between two points touching the lesion area when it is imagined that the wire lengthwise outline (61) is extended.

For example, the user may designate the measurement point (P) at an arbitrary position between A and B. When the user designates the wire lengthwise outline (61) and the measurement point (P), the image processing program (15) may seek by itself the points (A, B) where the wire lengthwise extension line (62) meets the lesion area in consideration of the measurement point (P), and may recognize a distance between two points (A, B) as a unit length (wire body diameter) on the image.

Furthermore, the user may input the wire body diameter connected by the biopsy tool to an actual length input item (213), whereby the image processing program (15) may recognize the scale through the calculated unit length on the image and the actual unit length thereto. Although the wire body diameter may be inputted by the user, the wire body diameter may be pre-set when the wire body diameter connected to the biopsy tool is standardized, or may be selected from pre-set several values.

FIG. 7 is a photographic image according to an exemplary embodiment of the present invention, showing a state where the wire lengthwise outline (61) inputted by a user passes a distal end (65) of the wire to be extended to a point (indicated by an arrow) where the lesion area is located, and the wire body diameter at this location is the unit length on the image. The actual unit length is set at 2.8mm, the value of which is a wire body diameter connected by the biopsy tool, and can be accurately set by the user.

The image processing program (15) can provide various drawing tools and functions by allowing drawing various desired shapes in round, oval, square, curved or polygonal shapes individually or in combination, whereby the user can designate the analysis object area as illustrated in FIG.4, or can conveniently perform a relevant operation when an out-boundary of the lesion area is directly designated, as illustrated in FIG. 5.

When the user clicks a calculation button (214-4), the image processing program (15) may calculate the size of the legion area based on the scale information obtained from S112 (S113).

The method for calculating the size of lesion area based on the scale may be variously configured. One of the methods for calculating the size of lesion area may include calculating by using the number of square lattices (the unit length on the image) entered into the lesion area. That is, when y number of square lattices each having a unit length on the image is entered into the lesion area, and an actual length corresponding to the unit length on the image is x, the size of the lesion area may be calculated as “X×X×Y”. Of course, when the square lattices at the out-boundary of the lesion area exist across an inside and outside of the out-boundary, only the inside part of the relevant square lattice is included to allow calculating an accurate size.

FIG. 8 illustrates an example where an inside of the out-boundary (81) of the lesion area designated by a user is filled with square lattices.

Meantime, the image processing program (15) may calculate a major axis length and a minor axis length of the legion area based on the scale information. The major axis and the minor axis may be defined by the longest axis and the shortest axis at a confirmed lesion area. The major axis and the minor axis are configured in a manner such that a distance to the farthest point from each point is measured along an outer circumference of the lesion area, where the farthest distance among the measured distances may be recognized as a major axis and the shortest distance may be recognized as a minor axis.

Furthermore, the major axis and the minor axis may be configured by being recognized through reception, from a user, of information designating a relevant axis. For example, as illustrated in FIG. 8, the user may click each distal point of the major axis, designate the major axis by drawing a line, or may click each distal point of the minor axis and designate the minor axis by drawing a line. Then, the image processing program (15) may calculate the actual length of the major axis and the minor axis using the scale information. The major axis length and the minor axis length may be used as information more accurately indicating the state of the lesion area, and may be used as basic data for calculating the size of the lesion area. For example, a square formed by a major length and a minor length may be simply confirmed as an image size of the lesion area.

The image processing program (15) may display on a screen the information on the calculated size of the lesion area, the major length and the minor length (S114).

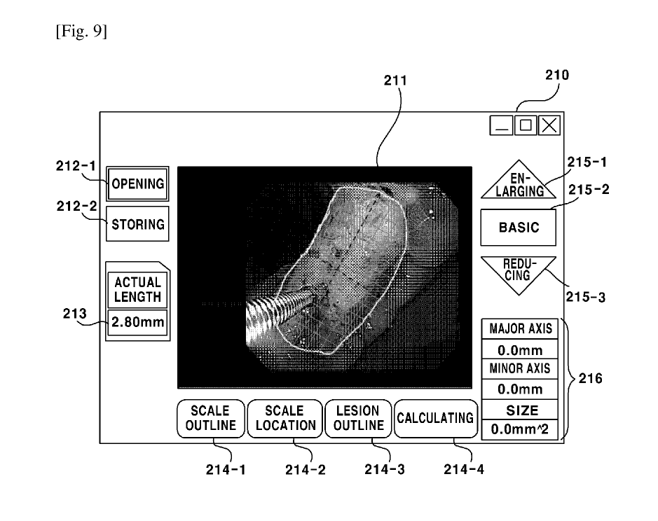

FIG. 9 is a schematic view illustrating a user interface screen displayed with information (216) on calculated size of legion area, major axis length and minor axis length according to an exemplary embodiment of the present invention.

The computer program configured to measure the size of lesion area according to each exemplary embodiment of the present invention thus described above may be executed by being loaded on a memory of a computer system.

The computer program may be pre-recorded in a computer readable storage medium such as hard disc drives or ROMs (Read-Only Memories). Furthermore, the computer program may be temporarily or permanently stored in a computer readable storage medium, such as, but is not limited to, any type of disk including flexible disks, optical disks, CD-ROMs, magnetic-optical disks, read-only memories (ROMs), DVD (Digital Versatile Disc), magnetic disc, semiconductor memory or any other type of media suitable for storing electronic instructions, and capable of being attached or detached to a computer system (10).

In addition, the computer program may be transmitted to the computer system from a download site, or transmitted to the computer system (10) via a network such as LAN (Local Area Network) or internet network, and the computer program received by the computer system (10) may be installed at a recording medium such as hard disc.

While the preferred embodiments of the invention are illustrated and described here, it is clear that the invention is not so limited. Numerous modifications, changes, variations, substitutions, and equivalents will occur to those skilled in the art having the benefit of this disclosure without departing from the spirit and scope of the present invention as defined by the following claims.

### INDUSTRIAL APPLICABILITY

The exemplary embodiments of the present invention have an industrial applicability in that size of the lesion can be accurately and quickly calculated from an image photographed by using a photographing device such as an endoscope, which is conducive to medical doctors in diagnosis and treatment.

1. A method for measuring size of lesion which is shown by endoscope, the method comprising:
displaying, on a screen, an image shown with a lesion which is shown by an endoscope while being performed by a computer program executed by a computer device;
determining a lesion area of the image displayed on the screen, and a scale; and
calculating a size of the determined lesion area using the scale.

2. The method of claim 1, further comprising displaying the calculated size of the lesion area on the screen.

3. The method of claim 1, wherein the determination of the lesion area is realized by color comparison.

4. The method of claim 3, wherein the color comparison is performed only within an analysis object area inputted by a user.

5. The method of claim 1, wherein the determination of the lesion area is performed based on an area inputted by a user.

6. The method of claim 1, further comprising receiving from a user information for calculating a unit length on the image.

7. The method of claim 6, wherein the image displayed on the screen is an image photographed along with a wire connected to a biopsy tool, and the computer program provides a function of allowing a user to draw an outline to the lesion area along a lengthwise direction of the wire, and a function of allowing the outline to designate two points touched by the lesion area, and recognizes a length between the designated two points as a unit length of the image.

8. The method of claim 1, wherein the size of the lesion area is calculated by using the number of square lattices entered into the lesion area, where length of each side is a unit length of the image.

9. The method of claim 1, further comprising calculating a major axis length and a minor axis length of the determined lesion area, or one of the major axis length and the minor axis length of the determined lesion area, based on the scale.

10. The method of claim 9, wherein major axis and minor axis are recognized by receiving information that designates a relevant axis.

11. A computer readable recording medium of claim 1,
wherein the computer readable recording medium is recorded with a program capable of executing the method for measuring size of lesion which is shown by endoscope.
